(19) United States
(12) Patent Application Publication
(10) Pub. No.: US 2005/0214222 A1
(43) Pub. Date: Sep. 29, 2005

(54) In vivo imaging of amyloid plaques in glaucoma using intravenous injectable dyes

(76) Inventors: Stuart J. McKinnon, Hillsborough, NC (US); Lorraine T. Kasmala, Durham, NC (US); Hank F. Kung, Wynnewood, PA (US)

Correspondence Address: MEYERTONS, HOOD, KIVLIN, KOWERT & GOETZEL, P.C., P.O. BOX 398, AUSTIN, TX, 78767-0398, US

(21) Appl. No.: 11/057,419

(22) Filed: Feb. 14, 2005

Related U.S. Application Data
(60) Provisional application No. 60/544,608, filed on Feb. 13, 2004.

(51) Int. Cl. A61K 49/00; G06K 9/00

(52) U.S. Cl. 424/9.6; 382/128

(57) ABSTRACT

In vivo imaging may be used to assess a condition (e.g., a state of glaucoma or a state of ocular hypertension) of an eye of a living animal. A dye may be intravenously injected into the living animal. The dye may bind to amyloid in the nervous system of the animal. Images may be taken of a retina, an optic nerve head, an optic nerve, the lateral geniculate nucleus, and/or the visual cortex. Images may be taken using methods such as fluorescent angiography, magnetic resonance imaging, computed tomography, positron emission tomography, and/or single photon emission computed tomography. The condition of the eye and/or retinal ganglion cells in the eye may be assessed from one or more of the images. The condition of the eye may be assessed based on the presence of amyloid in one or more of the images.

### PRIORITY CLAIM

This application claims the benefits of U.S. Provisional Patent Application No. 60/544,608 entitled “In Vivo Imaging Of Amyloid Plaques In Glaucoma Using Intravenous Injectable Dyes” to McKinnon et al., filed on Feb. 13, 2004.

### BACKGROUND

1. Field of the Invention

This invention relates generally to methods for in vivo imaging of retinal ganglion cells in the eye and associated portions of the nervous system. An embodiment of the invention may be used to detect ocular hypertension in the eye.

2. Description of Related Art

Glaucoma is an eye disease that gradually reduces the sight of an affected individual over time. Often, glaucoma will occur without obvious signs or symptoms. It is estimated that over 3 million Americans have glaucoma but that only about one-half of those have been diagnosed with the disease. Typically, ocular hypertension is a main cause of glaucoma, although other factors may be involved. Detection of glaucoma may involve a visual field test. Visual field testing, however, only detects damage from ocular hypertension or glaucoma after the disease has progressed to an advanced state. For example, current visual field tests may only detect damage after loss of about 30% to about 50% of the retinal ganglion cells (the cells damaged by glaucoma). Thus, an individual's sight may already be severely damaged by the time glaucoma is detected. Experiments indicate that amyloid is upregulated in retinal ganglion cells as the cells become damaged by ocular hypertension or glaucoma (see Stuart J. McKinnon “Glaucoma: ocular Alzheimer's disease?” Frontiers in Bioscience 8: 1140-1156 (Sep. 1, 2003), which is incorporated by reference as if fully set forth herein).

Amyloid plaques are currently used as a marker for detecting Alzheimer's disease. Amyloid plaques have been labeled with an intravenously injectable dye for detection of Alzheimer's disease. D. Skovronsky et al., “In vivo detection of amyloid plaques in a mouse model of Alzheimer's disease” PNAS 97(13): 7609-7614 (Jun. 20, 2000); C. Lee et al., “Dimethylamino-fluorenes: ligands for detecting β-amyloid plaques in the brain” Nuclear Medicine and Biology 30: 573-580 (2003); and M. Ono et al., “¹¹C-labeled stilbene derivatives as Aβ-aggregate-specific PET imaging agents for Alzheimer's disease” Nuclear Medicine and Biology 30: 565-571 (2003), each of which is incorporated by reference as if fully set forth herein, describe techniques and dyes used for detection of Alzheimer's disease. Amyloid has also been seen in the cataracts of Alzheimer's patients (see Goldstein L. E et al., “Cytosolic beta-amyloid deposition and supranuclear cataracts in lenses from people with Alzheimer's disease” Lancet 361(9365): 1258-65 (2003), which is incorporated by reference as if fully set forth herein). Drug research for Alzheimer's disease has provided some focus on developing drugs that inhibit or clear amyloid from the body. These drugs may also be useful for treatment of glaucoma or other ocular hypertension disorders.

### SUMMARY

In an embodiment, in vivo imaging is used to assess a condition of an eye of a living animal. A dye may be intravenously injected into the living animal. The dye may cross the blood-brain barrier of the animal. The dye may bind to amyloid in the nervous system of the animal. Images may be taken of one or more portions of the nervous system of the animal. Images may be taken using methods such as fluorescent angiography, magnetic resonance imaging, computed tomography, positron emission tomography, and/or single photon emission computed tomography.

A condition of the eye and/or retinal ganglion cells in the eye may be assessed from one or more of the images taken. The condition of the eye may be assessed based on the presence of amyloid in one or more of the images taken. The condition of the eye may include a disease state (e.g., a state of glaucoma or a state of ocular hypertension) of the eye.

In certain embodiments, images may be taken of a retina, an optic nerve head, an optic nerve, a lateral geniculate nucleus, and/or the visual cortex of the brain. In some embodiments, the condition of the eye may be assessed based on a quantitative measurement of amyloid detected in one or more of the images. Changes in the condition of the eye may be monitored over a period of time.

### BRIEF DESCRIPTION OF THE DRAWINGS

Advantages of the present invention may become apparent to those skilled in the art with the benefit of the following detailed description and upon reference to the accompanying drawings in which:

FIG. 1 depicts a schematic of an embodiment for injecting a dye into a human body.

FIG. 2A depicts structures for several imaging dyes.

FIG. 2B depicts structures for several imaging dyes.

FIG. 3 depicts structures for several imaging dyes.

FIG. 4 depicts structures for several imaging dyes.

FIG. 5 depicts structures for several imaging dyes.

FIG. 6 depicts a flowchart for imaging portions of a nervous system of an animal and assessing conditions in an eye of the animal.

FIG. 7 depicts a schematic view of a human eye and associated nervous system components.

FIG. 8 depicts an illustrative view of a human brain and eyes.

FIG. 9 depicts a schematic view of a human brain and the visual pathways of the brain.

While the invention is susceptible to various modifications and alternative forms, specific embodiments thereof are shown by way of example in the drawings and may herein be described in detail. The drawings may not be to scale. It should be understood, however, that the drawings and detailed description thereto are not intended to limit the invention to the particular form disclosed, but on the contrary, the intention is to cover all modifications, equivalents, and alternatives falling within the spirit and scope of the present invention as defined by the appended claims.

### DETAILED DESCRIPTION

In vivo imaging of a living animal (e.g., a human) may be used to detect and/or monitor conditions associated with disease in the animal. In certain embodiments, conditions associated with, for example, ocular hypertension may be detected and/or monitored in a living animal through in vivo imaging. Images of the eye of the animal and/or portions of the nervous system proximate to the eye may be used to assess conditions associated with disease in the eye. In certain embodiments, a dye may be injected into the animal to enable typical imaging techniques (e.g., fluorescent angiography, magnetic resonance imaging (MRI), computed tomography (CT), positron emission tomography (PET), single photon emission tomography (SPECT)) to be used for detection and/or monitoring of disease state in the eye of the animal. The dye may bind or attach to certain proteins or plaques of the animal that are indicative of disease within the eye.

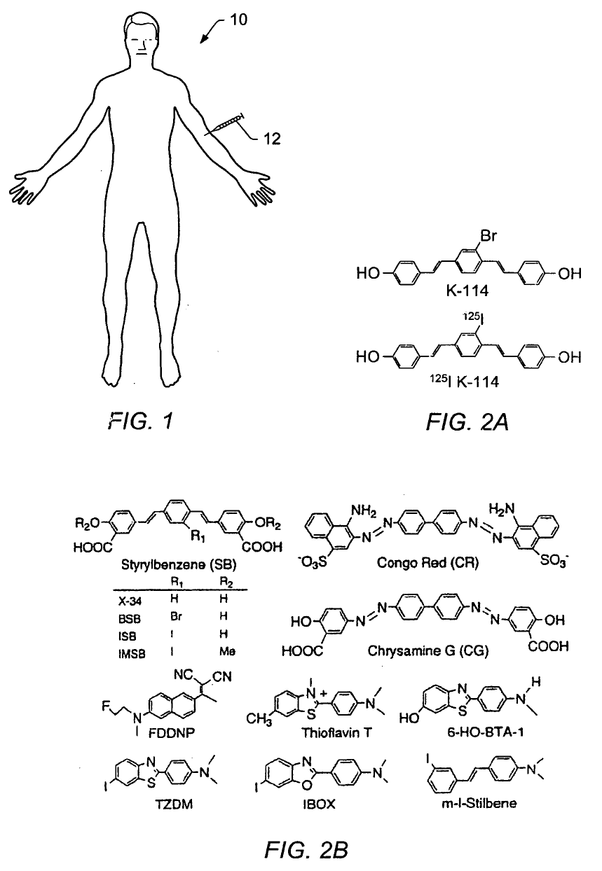

FIG. 1 depicts a schematic of an embodiment for injecting a dye into a human body. In certain embodiments, the dye may be injected intravenously. The dye may be injected using syringe 12 or other device suitable for introducing the dye into the circulatory system of human 10. The dye may be an intravenously injectable dye that that crosses the blood-brain barrier of human 10. The dye may bind or attach to cell plaques or proteins in the nervous system of human 10. The dye may bind specifically to (e.g., “label”) the plaques so that the plaques are identifiable in images taken of the nervous system by, for example, fluorescent angiography. The labeled plaques may result in a contrast in an image of the nervous system (e.g., the dye bound to the plaques may fluoresce during imaging).

In certain embodiments, an intravenously injectable dye binds to amyloid proteins or amyloid plaques in the nervous system. Amyloid may be an indicator of ocular hypertension (e.g., glaucoma) in the eye of an animal. Amyloid plaques have previously been used as a marker for Alzheimer's disease as shown by: D. Skovronsky et al., “In vivo detection of amyloid plaques in a mouse model of Alzheimer's disease”; C. Lee et al., “Dimethylamino-fluorenes: ligands for detecting β-amyloid plaques in the brain”; and M. Ono et al., “¹¹C-labeled stilbene derivatives as Aβ-aggregate-specific PET imaging agents for Alzheimer's disease”. Amyloid may be upregulated in retinal ganglion cells (RGCs) that are damaged due to ocular hypertension. Thus, amyloid may be detected in images of the eye or portions of the nervous system associated with the eye as an indicator of RGCs that have been damaged by ocular hypertension (i.e., RGCs undergoing neuronal degeneration due to ocular hypertension).

An intravenously injectable dye that binds to amyloid may be used for different types of labeling. The dye may be able to cross the blood-brain barrier in an animal or a human. In certain embodiments, a dye may be made to label the amyloid for a selected type of imaging method. For example, in one embodiment, the dye may allow detection of amyloid in images taken by fluorescent angiography. In some embodiments, the dye may allow detection of amyloid in images taken by MRI, CT, or positron emission tomography (PET). An example of an intravenously injectable dye is K-114 ((trans,trans),-1-bromo-2,5-bis-(4-hydroxy)styrylbenzene), shown in FIG. 2B. In some embodiments, a dye may be isotope-labeled (e.g., radiolabeled). Radiolabeling may be used to measure binding of the dye to the plaque or protein more quantitatively. For example, ¹²⁵I- or ¹²³I-radiolabeled K-114 may be used in single photon emission computed tomography. FIG. 2A depicts structures for K-114 and ¹²⁵I-radiolabeled K-114.

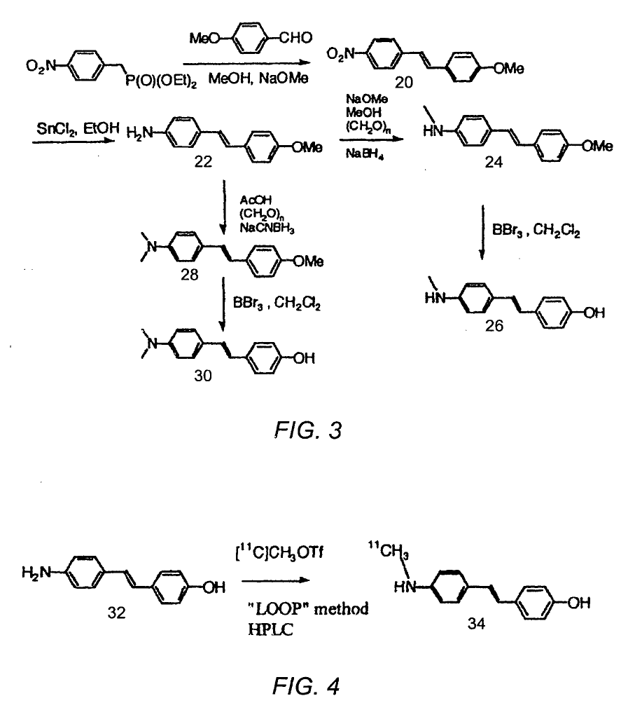

Benzothiazoles, stilbenes, styrylbenzenes, and their derivatives may be used as dyes for amyloid imaging. K-114 is one example of a styrylbenzene dye. Stilbene-based dyes may include two phenyl rings. One of the phenyl rings may include an electron-donating group such as, but not limited to, p-Me₂N—, —OMe, or —OH. In some embodiments, ^{99m}Tc, ¹²³I, ¹²⁵I, or ¹⁸F may be attached to a phenyl ring for radiolabeling. In certain embodiments, (trans,trans),-1-bromo-2,5-bis-(3-hydroxycarbonyl-4-hydroxy)styrylbenzene (BSB) described by D. Skovronsky et al. in “In vivo detection of amyloid plaques in a mouse model of Alzheimer's disease” may be used as a dye for amyloid imaging. BSB is a styrylbenzene dye. In certain embodiments, ¹²⁵I-radiolabeled BSB (ISB) may be used for quantitative measurements of the binding of BSB to amyloid. BSB and some other imaging agents are depicted in FIG. 2B. In some embodiments, extra halogens may be added to a phenyl ring of stilbenes to increase the potency of binding to amyloid. Some possible stilbene derivatives (molecules 20-30, listed below) are depicted in FIG. 3 and described by M. Ono et al. in “¹¹C-labeled stilbene derivatives as Aβ-aggregate-specific PET imaging agents for Alzheimer's disease”.

| Molecule No. | Molecule Name |
|---|---|
| 20 | (E)-4-nitro-4′-methoxystilbene |
| 22 | (E)-4-amino-4′-methoxystilbene |
| 24 | (E)-4-methylamino-4′-methoxystilbene |
| 26 | (E)-4-methylamino-4′-hydroxystilbene |
| 28 | (E)-4-Dimethylamino-4′-methoxystilbene |
| 30 | (E)-4-Dimethylamino-4′-hydroxystilbene |

In some embodiments, a dye may be radiolabeled with a carbon isotope (e.g., ¹¹C). For example, in FIG. 4, molecule 34 ([¹¹C](E)-4-methylamino-4′-hydroxystilbene) is a ¹¹C-radiolabeled derivative of molecule 32 ((E)-4-amino-4′-hydroxystilbene).

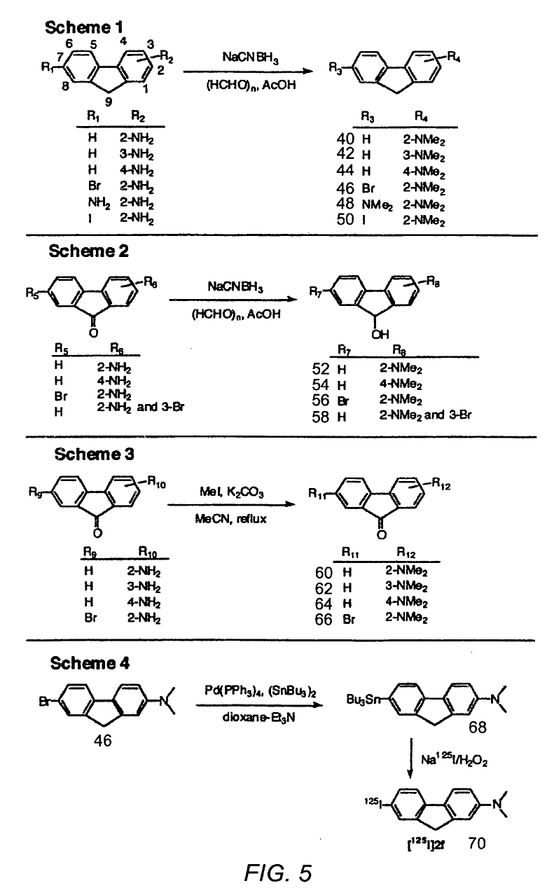

In certain embodiments, tri-cyclic fluorene derivatives may be used as dyes for amyloid imaging. N,N-dimethylamino derivatives of fluorene may be used as amyloid imaging dyes as described in C. Lee et al., “Dimethylamino-fluorenes: ligands for detecting β-amyloid plaques in the brain”. FIG. 5 depicts structures (molecules 40-68) for several N,N-dimethylamino-fluorene derivatives that may be used for amyloid imaging.

| Molecule No. | Molecule Name |
|---|---|
| 40 | 2-(Dimethylamino)fluorene |
| 42 | 3-(Dimethylamino)fluorene |
| 44 | 4-(Dimethylamino)fluorene |
| 46 | 2-Dimethylamino-7-bromofluorene |
| 48 | 2,7-Bis(dimethylamine)fluorene |
| 50 | 2-Dimethylamino-7-iodofluorene |
| 52 | 2-Dimethylamino-9-hydroxyfluorene |
| 54 | 4-Dimethylamino-9-hydroxyfluorene |
| 56 | 2-Dimethylamino-7-bromo-9-hydroxyfluorene |
| 58 | 2-Dimethylamino-3-bromo-9-hydroxyfluorene |
| 60 | 2-Dimethylamino-9-fluorenone |
| 62 | 3-Dimethylamino-9-fluorenone |
| 64 | 4-Dimethylamino-9-fluorenone |
| 66 | 2-Dimethylamino-7-bromo-9-fluorenone |
| 68 | 2-Dimethylamino-7-(tributylstannyl)fluorene |

In some embodiments, N,N-dimethylamino-fluorene derivatives may be radiolabeled with a halogen isotope (e.g., ¹²³I or ¹²⁵I) or a carbon isotope (e.g., ¹¹C). FIG. 5 depicts [¹²⁵I]7-iodo-2-N,N-dimethylamino-9-hydroxyfluorene (molecule 70), which may be formed from molecule 68.

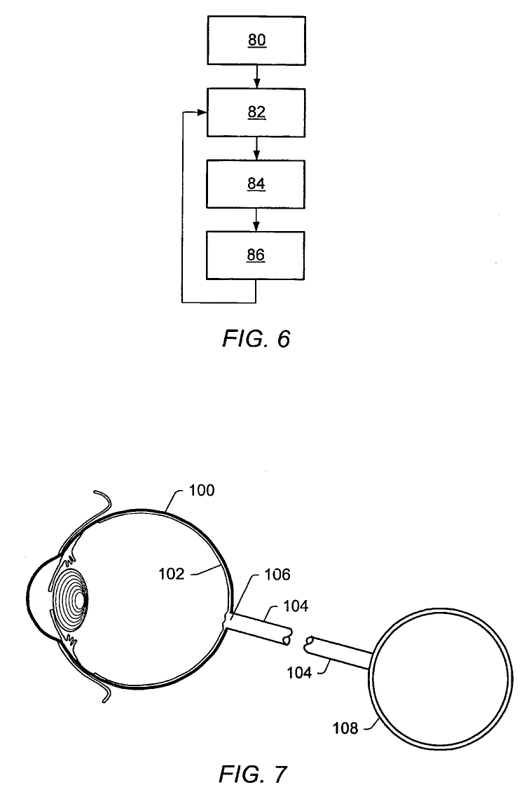

FIG. 6 depicts a flowchart for imaging portions of a nervous system of an animal and assessing conditions in an eye of the animal. During injection 80, the animal (e.g., a human or rat) may be injected with a dye. Following injection, the dye may enter the nervous system and bind to amyloid. After an elapsed time, images of the nervous system may be obtained. Imaging 82 may include imaging one or both eyes of the animal and portions of the nervous system associated with each eye (e.g., the optic nerve, lateral geniculate nucleus, or visual cortex). Imaging 82 may be performed using an imaging technique such as, but not limited to, fluorescent angiography with fundus photography, MRI, CT, PET, or SPECT. Following imaging 82, assessing 84 may include using images taken during the imaging to assess a condition (e.g., a disease state) of the eye. The condition may include a state of ocular hypertension or a state of glaucoma in the eye.

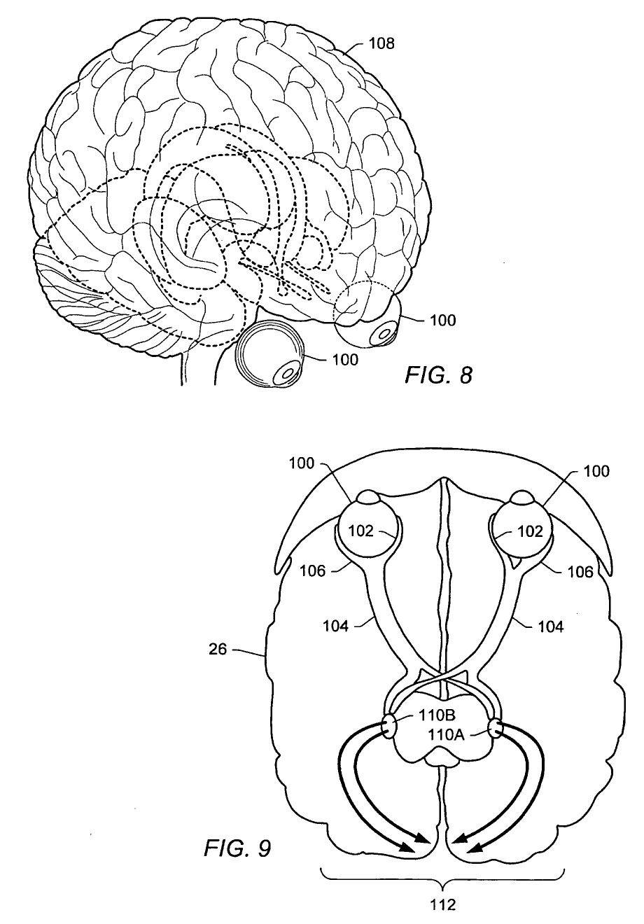

FIG. 7 depicts a schematic view of an eye and associated nervous system components. Eye 100 includes retina 102. Optic nerve 104 is attached to eye 100 at optic nerve head 106 and connects the eye to brain 108. FIG. 8 depicts an illustrative view of brain 108 and eyes 100. Images may be taken of each eye 100 (left or right), each optic nerve head (left or right), each optic nerve (left or right), and/or one or more portions of brain 108 associated with the eyes (e.g., the lateral geniculate nucleus and/or the visual cortex).

FIG. 9 depicts a schematic view of a human brain and the visual pathways of the brain including lateral geniculate nucleus 110 and visual cortex 112. Lateral geniculate nucleus 110 is the target of the RGC axons in humans. Right lateral geniculate nucleus 110A is associated with uncrossed RGC axons from the temporal visual field of the right eye and crossed RGC axons from the nasal visual field of the left eye. Left lateral geniculate nucleus 110B is associated with uncrossed RGC axons from the temporal visual field of the left eye and crossed RGC axons from the nasal visual field of the right eye. Secondary neurons project from the lateral geniculate nucleus to visual cortex 112. Thus, images of retina 102 may show amyloid in damaged or diseased eyes. Secondary neuronal degeneration may be seen in optic nerve 104, lateral geniculate nucleus 110, and/or visual cortex 112. Images of optic nerve head 106, optic nerve 104, lateral geniculate nucleus 110 and/or visual cortex 112 may indicate labeling of amyloid.

Amyloid may accumulate in primary and/or secondary targets of RGC axons in the retina (e.g., the optic nerve head, the optic nerve, the lateral geniculate nucleus, and/or the visual cortex). In primates, about 50% of RGC axons in one eye may target to the lateral geniculate nucleus of the same side (e.g., about 50% of the RGC axons of the right eye target the right lateral geniculate nucleus). Thus, image detection of amyloid in the right (left) lateral geniculate nucleus, the right (left) visual cortex, the right (left) optic nerve head, and/or the right (left) optic nerve may be attributed to ocular hypertension in either eye. In early stages of ocular hypertension, amyloid may be detected primarily in the retina. As the disease advances, the optic nerve head, the optic nerve, the lateral geniculate nucleus, and/or the visual cortex may become affected and show amyloid in images. During advanced stages of ocular hypertension, loss of RGCs in the retina may result. Image detection of amyloid primarily in the optic nerve head, the optic nerve, the lateral geniculate nucleus, and/or the visual cortex may be an indicator of advanced ocular hypertension. Generally, the relative amounts of amyloid in the portions imaged (i.e., the retina, optic nerve head, optic nerve, lateral geniculate nucleus, and/or visual cortex) may be correlated to a level or state of neuronal degeneration.

In FIG. 6, imaging 82 is followed by assessing 84. In certain embodiments, assessing 84 may include determining the extent of neuronal degeneration in the retina. Neuronal degeneration may be the result of ocular hypertension or glaucoma. The amount of damage caused by ocular hypertension is correlated to the level or state of neuronal degeneration, which may be represented by the relative amounts of amyloid detected in images of the eye and its associated components. In the early stages of ocular hypertension or glaucoma, small amounts of amyloid may be detected in images taken using techniques described herein. Image detection of small amounts of amyloid may provide earlier detection of damage or neuronal degeneration caused by ocular hypertension or glaucoma than currently used visual techniques. Current visual techniques typically detect damage only after about 30% to about 50% of RGCs are destroyed due to ocular hypertension. Imaging of amyloid may detect damage due to ocular hypertension before about 30% or less of RGCs are destroyed. In some cases, imaging of amyloid may detect damage due to ocular hypertension before any RGCs are destroyed.

In FIG. 6, monitoring 86 may include repeating imaging 82 and assessing 84 neuronal degeneration over a period of time. Neuronal degeneration (e.g., the loss of RGCs due to glaucomatous damage) may be monitored as a function of amyloid exposure in images taken over time. In some embodiments, images of the retina, the optic nerve head, the optic nerve, the lateral geniculate nucleus, and/or the visual cortex may be monitored to follow the state of disease (e.g., ocular hypertension, glaucoma, or other chronic neurodegenerations) over time. For example, the relative amounts of amyloid in the retina, the optic nerve head, the optic nerve, the lateral geniculate nucleus, and/or the visual cortex may be monitored. In certain embodiments, the incremental loss of RGCs may be recorded, as generally shown by the presence of amyloid in the retina.

In some embodiments, individual RGCs may be imaged. For example, adaptive optic systems used to image individual photoreceptor cells may be used to image RGCs with a bound dye that demonstrates a quantum yield. An intravenous injectable dye bound to amyloid may provide a quantum yield measurable by an imaging technique. Imaging individual RGCs may be used to generate a map of RGCs in a retina. More than one map of individual RGCs may be generated over time. A disease state of the eye may be monitored using the maps of individual RGCs. In some embodiments, a disease state of the eye may be monitored longitudinally using the maps of individual RGCs. In some embodiments, a radiolabeled dye may be used to allow quantitative measurement of the binding between amyloid and the dye.

In this patent, certain U.S. patents, U.S. patent applications, and other materials (e.g., articles) have been incorporated by reference. The text of such U.S. patents, U.S. patent applications, and other materials is, however, only incorporated by reference to the extent that no conflict exists between such text and the other statements and drawings set forth herein. In the event of such conflict, then any such conflicting text in such incorporated by reference U.S. patents, U.S. patent applications, and other materials is specifically not incorporated by reference in this patent.

Further modifications and alternative embodiments of various aspects of the invention will be apparent to those skilled in the art in view of this description. Accordingly, this description is to be construed as illustrative only and is for the purpose of teaching those skilled in the art the general manner of carrying out the invention. It is to be understood that the forms of the invention shown and described herein are to be taken as the presently preferred embodiments. Elements and materials may be substituted for those illustrated and described herein, parts and processes may be reversed, and certain features of the invention may be utilized independently, all as would be apparent to one skilled in the art after having the benefit of this description of the invention. Changes may be made in the elements described herein without departing from the spirit and scope of the invention as described in the following claims.

1. A method for in vivo imaging, comprising:
intravenously injecting a dye into the circulatory system of a living animal, wherein the dye is configured to bind to amyloid in one or more portions of the nervous system of the animal;
obtaining one or more images of at least one of the portions of the nervous system of the animal comprising the dye; and
assessing a condition of an eye from at least one of the images.

2. The method of claim 1, further comprising assessing a condition of retinal ganglion cells in the eye from at least one of the images.

3. (canceled)

4. The method of claim 1, wherein at least one of the portions of the nervous system comprises a retina of the eye and portions of the nervous system associated with the eye.

5. The method of claim 1, wherein at least one of the portions of the nervous system comprises an optic nerve head coupled to the eye.

6. The method of claim 1, wherein at least one of the portions of the nervous system comprises an optic nerve coupled to the eye.

7. The method of claim 1, wherein at least one of the portions of the nervous system comprises at least part of the lateral geniculate nucleus.

8. The method of claim 1, wherein at least one of the portions of the nervous system comprises at least part of the visual cortex.

9. The method of claim 1, wherein at least one of the portions of the nervous system comprises retinal ganglion cells.

10. The method of claim 1, further comprising allowing the dye to bind to amyloid in at least one of the portions of the nervous system.

11. The method of claim 1, wherein the dye is configured to cross the blood-brain barrier.

12-18. (canceled)

19. The method of claim 1, wherein the condition comprises a state of glaucoma in the eye.

20. The method of claim 1, wherein the condition comprises a state of ocular hypertension in the eye.

21-25. (canceled)

26. The method of claim 1, further comprising assessing the condition of the eye based on a quantitative measurement of amyloid detected in at least one of the images.

27. (canceled)

28. The method of claim 1, further comprising monitoring changes in the condition of the eye over time, wherein changes in the condition of the eye are represented by a quantitative measurement of amyloid detected in at least one of the images.

29. The method of claim 1, further comprising obtaining a quantum yield of the dye bound to the amyloid.

30. The method of claim 1, further comprising generating one or more maps of retinal ganglion cells comprising amyloid.

31. The method of claim 30, further comprising monitoring disease state of the eye using one or more of the maps of retinal ganglion cells.

32. The method of claim 30, further comprising longitudinally monitoring disease state of the eye using one or more of the maps of retinal ganglion cells.

33-64. (canceled)

65. A method for assessing a disease state of an eye of a living animal, comprising:
intravenously injecting a dye into the circulatory system of the animal, wherein the dye is configured to bind to amyloid;
obtaining one or more images of one or more portions of the nervous system of the animal comprising the dye, wherein the portions are part of the eye of the animal or are proximate to the eye of the animal; and
assessing the disease state of the eye by assessing the presence of amyloid in the images.

66-68. (canceled)

69. The method of claim 65, wherein at least one of the portions of the nervous system comprises an optic nerve head coupled to the eye.

70. The method of claim 65, wherein at least one of the portions of the nervous system comprises an optic nerve coupled to the eye.

71. The method of claim 65, wherein at least one of the portions of the nervous system comprises at least part of the lateral geniculate nucleus proximate to the eye of the animal.

72. The method of claim 65, wherein at least one of the portions of the nervous system comprises at least part of the visual cortex proximate to the eye of the animal.

73-89. (canceled)

90. The method of claim 65, further comprising assessing the disease state of the eye based on a quantitative measurement of amyloid detected in at least one of the images.

91. (canceled)

92. The method of claim 65, further comprising monitoring changes in the disease state of the eye over time, wherein changes in the disease state of the eye are represented by a quantitative measurement of amyloid detected in at least one of the images.

93-96. (canceled)

97. A method for in vivo imaging of retinal ganglion cells, comprising:
intravenously injecting a dye into the circulatory system of a living animal, wherein the dye is configured to bind to amyloid in one or more portions of the nervous system of the animal; and
obtaining one or more images of retinal ganglion cells comprising amyloid in a part of an eye of the animal.

98. The method of claim 97, further comprising assessing a condition of retinal ganglion cells in the eye from at least one of the images.

99. The method of claim 97, wherein the part of the eye comprises a retina of the eye.

100-117. (canceled)

118. The method of claim 97, further comprising assessing a state of glaucoma in the eye.

119. The method of claim 97, further comprising assessing a state of ocular hypertension in the eye.

120-124. (canceled)

125. The method of claim 97, further comprising assessing a condition of the eye based on a quantitative measurement of amyloid detected in at least one of the images.

126-131. (canceled)
